(19) United States
(12) Patent Application Publication
(10) Pub. No.: US 2020/0168313 A1
(43) Pub. Date: May 28, 2020

(54) MEDICAL ENGINEERING APPARATUS FOR STORING AND EVALUATING CLINICAL DATA

(71) Applicant: W.O.M. WORLD OF MEDICINE GMBH, Berlin (DE)

(72) Inventor: Peter WESTERHOFF, Berlin (DE)

(21) Appl. No.: 16/632,906

(22) PCT Filed: Jul. 10, 2018
(86) PCT No.: PCT/DE2018/000211

§ 371 Date: Jan. 22, 2020

(30) Foreign Application Priority Data
Jul. 28, 2017 (DE) 10 2017 007 099.7
Dec. 29, 2017 (DE) 10 2017 012 064.1

(51) Int. Cl. G16H 20/40; G16H 10/60; G16H 40/63

(52) U.S. Cl. CPC G16H 20/40; G16H 40/63; G16H 10/60

(57) ABSTRACT

Subject matter of the invention is a medical engineering apparatus for storing medical data for information collection, information evaluation, and iterative information-based process optimization, which includes an automatic knowledge accumulation and provision by an automated and anonymized acquisition of data of networked components of the operating room for the minimally invasive surgery (MIS), i.e., e.g., insufflators, pumps, cameras, monitors, therapy instruments as well as by assessments of the post-operative treatment success linked with these data.

### FIELD OF THE INVENTION

Subject matter of the invention is a device for utilizing and providing data from networked medical devices for a device configuration tailored to patient's individual needs and for disburdening the work process.

Herein, “networking” means the comprehensive use of information of all relevant data present in the operating room. A “medical device” is every energy-operated or non-energy-operated technical device that is used for diagnostic or therapeutic interaction with the human or animal body. Accessories and disposables are explicitly also referred to as medical devices.

### PRIOR ART

Currently, information about the surgery process and the recovery process of the patient is not supplied to a central pool of experiences and is thus not available as a general base of knowledge for future interventions, nor can it automatically be evaluated. Technical process parameters are rarely analyzed or evaluated in the context of the clinical result. The experiences about performed surgeries are not sufficiently evaluated, documented and published, not even in publications.

In medical guidelines, the medical actions are laid down, evidence-based by clinical studies, with a great time delay for diagnostic and/or therapeutic procedures. These guidelines are rather not detailed enough as consensus papers of medical expert associations for the settings of medical devices or technical parameters for deriving device settings. With respect to the setting of operating parameters of the medical devices, the manufacturers of medical devices have advanced knowledge, in part imparted in training sessions. All these described ways of a standardized application of medical devices for a diagnostic or therapeutic procedure for good clinical results for the patient are time-consuming and are accompanied by great efforts.

Furthermore, the complex relations of device settings with the clinical treatment success are often not investigated in studies, since the documentation of the device parameters can in most cases not be performed by the medical personnel involved in the study. Set values are introduced manually by the medical personnel based on individual experiences.

EP 1995679 describes a device and a method for networking and central operation and data transmission for documentation purposes and/or control or parameter adjustment of at least one device that is used during a medical intervention. A central database is provided, which is kept on a control unit, an external server accessible via networks or a readable data carrier. Paragraph [0030] describes the access to the central database via a local network or the internet.

“Desirably,” this database contains, according to paragraph [0008], expert knowledge and/or predetermined device parameters being independent from the respective medical personnel, said device parameters being extended by the device assembly mentioned in paragraph [0013] for the respective medical procedure and being suggested to the surgeon, i.e. a kind of assistance system for instrumentation. Paragraph [0023] describes that predetermined device configurations and parameters may be part of the database.

Paragraph [0016] describes that device parameters or device actions are initiated by a signal automatically generated by a sensor. There is, however, no technical teaching, except that a device presetting is automatically adopted—due to which sensors and what these will effect is not set forth. A regulation of device parameters by sensor values, in particular, how the solution according to the invention illustrated herein is embodied, is not described and is not obvious.

Each acquisition of the database contents in a device is linked, in EP1995679, mandatorily with an approval of the surgeon, i.e. the actuation of a confirmation element.

Paragraph [0028] describes that data of the patient (age, height, weight, existing or already recognized diseases, anatomic peculiarities) require altered parameter values, device configurations, and optimum parameters depend on the employed devices and manufacturers. In paragraph [0029], the provision thereof in the database and different patient-dependent data sets or a formula for calculating patient-dependent parameters are described. As a technical teaching of a calculation, the interpolation or extrapolation is mentioned as an example. The solution according to the invention provides an evidence-based approach, i.e. this is also not described nor is it obvious.

EP 2763064 A2 describes an insulin pump with a configurator for setting the operating parameters of the insulin pump.

### SOLUTION ACCORDING TO THE INVENTION

For the solution of the defined problems, according to the invention, the acquisition and analysis of data in the medical engineering apparatus according to the present claims is achieved by adoption from the intra-operative phase and acquisition of new data in the post-operative phase.

The invention, therefore, relates to a medical engineering apparatus for storing medical data, including
at least one computation unit, consisting of a selection and correlation module, a feed module with self-learning sub-module,
at least one storage unit that stores the measurement data obtained via the interface and/or patient data and that optionally permanently includes at least one data set as a fall-back level,
at least one interface to at least one other medical engineering apparatus, the interface transmitting measurement data and/or patient data to the storage unit,
at least one interface for reading patient information to at least one information system of the hospital (patient record) and/or an image recognition system for reading the patient information,
at least one processing device, in which a priori information present in the storage unit is processed with information obtained via the interface such that operating parameters calculated according to a predetermined criterion are provided via an interface to another medical engineering apparatus.

In another step, from the currently transmitted data for already known scenarios that are supported with previously collected data, a derivation of device configurations or device settings can occur in the pre-operative phase by matching the patient data, and hints for the treatment from the intra-operative data according to the guidelines can be transmitted to the medical device.

A precondition is the provision of data interfaces to information sources in the operative environment such as the hospital information system (HIS) or the anesthesia system, as well as the specification of relevant information sources and interfaces. These interfaces may be embodied electronically or also otherwise, e.g., by image recognition and evaluation of a medical device with a camera of data on forms or screens.

The field of use of the expert system may comprise further functional units in the operating room. For example, as most important section, the anesthesia strongly benefits from a system of experiences continuously updating itself and the couplings to other clinical devices and systems.

The expert system is a device for acquisition of data from medical devices and pre- and post-operative patient data that links the patient data with process data determined during the procedure as well as clinical successful treatments and in doing so secures the association of the data with each other, consolidates such data groups in classes and combines the process data and sequence information with the best clinical results and continuously weighs them with new data input to each other, determines the process data with the best clinical result of a class as an optimum and then transmits the determined optimum back to the medical device as a pre-setting. When transmitting back, clinical guidelines and clinical experience-based knowledge with respect to the data groups and selection rules are included. The medical device further has a data fall-back level when the data transmission fails.

Automated acquisition and feedback of device configurations takes place by a central data collection, data analysis and data storage device, a so-called expert system that is accessible from the medical device via a network. This expert system consists of a selection and correlation module, a feed module for rules and expert knowledge with a self-learning sub-module and a data retention module, which assigns, classifies, stores the contents and manages the access to the contents. The contents are data sets consisting of anonymized patient data, anamnesis data, procedure information, information about employed medical devices, intra-operative vital data and device data or device parameters, post-operative treatment assessments. Furthermore, procedural rules are recorded in the expert system that are applied in the selection and correlation module. As an embodiment, a server may be accessible via the internet and only send back parameter characteristics to the device that provides the original information (device parameters, vital data, patient data).

By the central provision, e.g., a change of guidelines or adaptations to changed procedure sequences can easily be entered. Furthermore, a data set can be provided from as many different data sources as possible, thereby no arbitrary pre-selection (bias) taking place and a large database being created.

The concept of an experience and knowledge database updating itself with each intervention, which is made available for each surgeon, leads to a continuous optimization of the minimally invasive surgery.

By the linkage of the information from the expert system to the data from anamnesis and device recognition, device configurations can automatically be selected. These settings can be optimized to the respective therapy as per patient's individual needs and thus improve pre-operatively already the minimally invasive surgery. The same applies to set value inputs such as the pressure at the surgery site.

The solution according to the invention for the expert system includes at least a
data communication layer for networking of the devices in the surgery,
data classification or grouping of patient data from the pre-operative phase,
determination or transmission of medical devices and assemblies of medical devices used for the medical procedure,
determination and transmission of set value recommendations based on a priori knowledge to the medical device.

Furthermore, the solution according to the invention may include
identification method for automatic recognition of employed devices in the pre-operative phase,
determination and transmission of a set of characteristic curves for controller pre-setting based on a priori knowledge to the medical device,
data transmission from the intra-operative phase to the database system,
determination and transmission of a set of characteristic curves for controller pre-setting based on intra-operative data to the medical device,
determination and transmission of set value recommendations based on intra-operative data to the medical device,
data transmission from the post-operative phase to the database system,
entering rules originating from the medical expert knowledge or guidelines.

The automated information process analyzes and classifies patient and device data and hands them over to the database for further processing and recording. The classification process corresponds to the established method, and a suitable classificator structure is created for each field of application. Training and validation of the classificator is made by means of data from the clinical environment.

The database uses data from established medical evaluation methods, in order to assess the operative result (clinical outcome). An analysis device using process data of the database from the pre- and intra-operative phase and being anonymized links data sets of the expert system to data sets of previous interventions and newly collected data about the course of the surgery and the clinical result and identifies successful combinations. Entering medical expertise in the knowledge database provides recommendations for action in defined clinical scenarios, which are based on the current medical guidelines. This knowledge database is combined with the expert system that is supplied from the statistical evaluation of current and previous interventions. Thus, the information basis for a continuously learning assistance system is created, which is available for the optimization of future interventions.

Information from the intra-operative phase is collected, anonymized and analyzed, in order to construct an expert system in the post-operative phase. For this purpose, the following is performed in the post-operative phase, inter alia:
evaluation of the operative results (post-operative clinical outcome or clinical treatment success),
statistical evaluation and clustering of anonymized clinical data,
acquisition of medical recommendations for action from expert knowledge,
feedback of experiences to the device pre-settings,
in addition to the use of existing information, process information is collected, analyzed and provided for other networked medical partial systems.

Data classes (e.g., type of patient, type of surgery) or groups of data sets are created, and the respective patient and anamnesis data are analyzed. From the data analysis, set value recommendations and/or controller pre-settings of the medical devices are derived, by that from the data sets of same data classes provided with positive clinical successful treatments with respect to patient and procedure, according to pre-determined rules, the device parameters included in the data sets are brought together and these are transmitted back, as a recommendation or device configuration, via the network to the medical device.

Such a pre-determined rule could be to prefer an as low set pressure value as possible for the body cavity as a transmitted-back device parameter. Another rule could be an age-dependent set pressure value that is fixedly pre-determined. These rules are to be designed according to the medical requirements and risk assessment. The data sets are continuously collected. Transmitted-back device settings can be altered by the surgery team. In addition to the device settings directly responding to vital data, presets can be used, which determine device pre-settings or controller behavior.

During the automated updating of the expert system, the course of the therapy is quantified, and relevant aspects are recorded. By the feedback of experience-based knowledge from the follow-up to future surgeries, a digital care chain is created. Herein, an automated optimization of the minimally invasive surgery is started.

According to the invention, origin and regulation parameters of insufflators and pumps in the minimally invasive surgery in the medical device are changed directly or indirectly by data input, thereby an improved device performance being guaranteed, which improves the vision in the surgery site, reduces bleedings, and/or minimizes the burden of the blood circulation of the patient.

By the linkage of all pre-, intra- and post-operative patient and surgery information suggested as the solution according to the invention, the surgeon or the clinical personnel can be disburdened of technical configuration procedures of the employed devices.

Thus is obtained
an improvement of the therapy by classification and use of a priori knowledge and experiences,
a quantification of the treatment success,
an acquisition of expert knowledge,
an improvement of future interventions by feedback of experience values and expert knowledge in a database,
by means of
methods for automatic data processing and classification,
methods for a priori controller setting and set value recommendation,
methods for automated evaluation and assessment of clinical data,
iterative optimization of the MIS by automatic accumulation of experiences.

Anamnesis data are detected and recorded before every surgery. The use of these a priori data is made, up to now, only manually by the clinical personnel. An automated evaluation by classification methods is to be investigated, in order to be able to better adjust the therapy to the individual patient.

All a priori data from the pre-operative phase and the experience and knowledge database according to the invention are to be employed for an automatic device configuration. Therein, the device configuration is to take place automatically in the form of controller parameters and the set value input.

The solution according to the invention also includes a holistic optimization of process sequences by the linkage of electronically existing data with the regulation of quality-relevant process parameters for optimization of the device settings during the surgery process with the data of an already performed surgery recorded in the databases in time before and after. The basic idea of the invention is the iterative optimization of the MIS procedures by data collection.

According to the invention is provided an automated anamnesis evaluation that includes a fusion of current patient data with an expert system. The device systems used during the intervention are to be automatically recognized by means of identification methods. Furthermore, recommendations for action for the surgeon are generated from all available a priori data.

The transmission of patient and anamnesis data is not only patient-specifically, but case-specifically assigned to the intra-operatively collected data, i.e. referred to the period of time and the performed clinical procedure, and this data set in turn is assigned to the post-operatively determined quantitative assessments of the clinical treatment success and/or occurring impairments or complications.

The transmission of the data can be made automatically without intervention of a human user or with initiation of a human user or manually by entering into a data input mask by a human user.

The feedback of device settings to the medical device can be made through a previously established safe connection or as an addressed and encrypted data packet with defined target indication. The reception of the data packet can be acknowledged by the receiving medical device named as target.

Certain procedures that are caused by the medical course or for other reasons require a certain reaction of the medical device, can as a rule be recorded in the evaluation module of the expert system. These may be, for example, a reduction of the joint pressure being after a defined time above a reference value, rinsing (exchange of media) of the expanded body cavity after a defined period of time or more complex situations, such as the warning after a certain pressure/time combination (where longer time is put on a level with low pressure, shorter time with high pressure, and a maximum pressure/time limit should not be exceeded) or the gradual reduction of the set pressure with proceeding duration of the procedure or after an adjusted increase above a defined threshold. Further rules are imaginable and can be applied through the device parameters of the server sent from the medical device and answered with device settings adjusted to the incoming data.

Same as the patient and anamnesis data, the post-operative assessments of the treatment success are also fed back patient- and case-specifically into the expert system and assigned to the other data. The assignment can be made, according to prior art, in an anonymized manner through order features that are generated prior to this (e.g., a case number of the hospital) or through the time (date) and patient data (name, date of birth, and/or health insurance number) or through specially generated assignment features (random token).

For the implementation of a central expert system it is of course important that for medical devices even with missing connection prior to the setting into operation and in case of a failure of a network convection or interference, the operation will proceed in a safe condition. If a connection to the server is not available or the data transmission is interrupted in the course of the procedure, a fall-back level is activated in the medical device, which secures a safe condition for the operation of the medical device. Herein, certain quantities are maintained, if applicable, other quantities are changed to a safe mode.

For this purpose, the logic systems for control or regulation up to now implemented in the medical devices are held further active, and by the possibility of inputting via the network interface, the function of the logic system for control or regulation is not rendered inoperative. This can be achieved by that the supplied values remain maintained and always secure a safe condition, or by that a failure of the connection is detected and the altered values are changed to a safe value or a mixture of both.

Equally, with data incompletely transmitted to the central expert system according to the invention, a back-supply of such data can take place that guarantee a safe operation of the medical device.

Based on a supply of data about the medical procedure coming-up and/or the patient to be treated to the expert system, recommendations for action are forwarded to the clinical personnel by an assistance system according to the invention and/or hints to critical conditions are given.

Set value recommendations derived from the data analysis and/or controller pre-settings or a selection for control algorithms of the medical devices are incorporated in an assistance system for supporting the clinical personnel. The assistance system supports the surgeon by the linkage of an expert system continuously expanding with data sets of individual patient data and technical measurement values when optimum clinical treatment results are achieved.

The assistance systems can also derive the device settings suitable for certain used medical devices from the information of the employed or detected medical devices in the surgery and transmit them back to the requisitioning medical device. Thereby, experience-based knowledge can automatically be retrieved.

By the networked provision of device parameters and device data, outside of the device, recognition of certain critical conditions can automatically be performed, and a secured device configuration or settings can be recommended to the surgeon in the operating room and/or the user of the device, which can be rejected or accepted by pressing a button. An automated adoption is also imaginable, however, this requires higher efforts of risk management or validation of the function.

Further assistance functions may reside in a trend analysis of values generating a warning and thus modifiying the further progress compared to the determined trend by reaction of the surgery team or changed device settings.

In addition, a visualization of processes as assistance is imaginable, i.e., graphical courses of device parameters with inserted ideal course or border areas.

Another aspect of the linkage of an expert system is the acquisition of data that can be provided to the hospital for documentation of the course of the individual surgery or evaluated as quality assurance, used in an anonymized form for medical studies, or used in an anonymized form as a basis for the development of controllers for medical devices.

A pre-sorting process based on clinical expertise is to subdivide the anamnestic data into data classes (e.g., in the simplest case, type of patient, type of surgery or procedure, medication, etc.).

The patient therapy is significantly improved, for example, by that in medical procedures in the MIS, bleedings are reduced and/or undesired tissue damages by a too high pressure at the surgery site are prevented. The limits are difficult to determine, can however easily be determined from procedures with good to very good treatment success following the law of large numbers. This procedure is known in medicine as “evidence-based”. The law of large numbers is necessary, since every patient responds in a different way to the interventions and therapeutic measures, and therefore a spread will result. By the combination and classification, many collected data sets brought together to one type can be considered, and in this group or class, the device parameters can be analyzed and correlated with the quantified treatment success. A pure correlation would presumably generate false values, and therefore it is provided that an additional rule-based selection can take place. The expert system determines the optimum device settings by means of a statistical evaluation of the surgery data and of the determined surgery evaluations and feeds these back to the medical device. Thus, a relationship between type of patient and type of surgery, the execution of the intervention and the obtained result is established. The statistical evaluation is made based on already anonymized or pseudonymized data. The collection and evaluation of information and the information-based process optimization can be implemented in an anonymized manner and nevertheless tailored to patient's individual needs. For this purpose, for example, methods of the multivariate regression analysis are employed, but also other suitable methods. By means of these methods, an interface between the post- and pre-operative phases is created. The automated linkage of pre-operative knowledge to the intra-operative intervention under automated post-operative evaluation of surgery courses includes the risk of individual overfittings. The determination of data and model structures as well as the non-linear optimization is made according to prior art, e.g., according to the activities of or.net. The result is, however, always dependent on the chosen structure of the used data set, where in particular there is a risk of overfitting. The medical consequences derived from the automated evaluation have to be validated and made available for all following operative interventions. This occurs by an expert system superimposed to the process of the MIS. Starting from this system, the course of an intervention can also be planned and monitored.

An assessment of an operative intervention can be made under different aspects. In part, subjective impressions have a high significance. An objective automated evaluation method is required, which forms the basis of the data for the learning database that is part of the expert system according to the invention.

When deriving recommendations for action from an expert system, it is mandatorily necessary to identify a correlation between relevant clinical process parameters and a clinical result. The challenge is to derive causality from correlations. When this is not successful, there is the risk to derive false recommendations for action from the experience and knowledge database.

The employed standard computer systems are currently characterized by a strict separation of the real and virtual world. Future MIS system solutions are characterized by intelligent surgery units with networked system components, intelligent sensors and actuators. The embedded systems widely used today do not meet these requirements. A paradigm change to dedicated systems for this feedback and assistance functions is required.

According to the invention, an iteratively growing experience and knowledge database is provided that can be made available to the surgeon as an assistance system during every intervention in the field of the minimally invasive surgery. This concept offers a high potential for further fields in the clinical environment and is to be used for such fields as anesthesia, rehabilitation therapy, or the field of dialysis.

The medical engineering apparatus according to the invention consists, in one embodiment, of a central computer that can exchange measurement data of sensors, information about the patient treated with the medical device, and operating parameters of the medical devices via an interface with the network of the hospital, for example a safe network connection to medical devices in the surgery area. These medical devices are also medical engineering apparatus. This computer is connected to a storage unit, where data associated with a treatment case are collected and recorded in a structured manner. These data per treatment case include, in addition to the pseudonymized patient data, the transmitted technical operating data of the medical device as well as other transmitted data, e.g., of other connected medical devices, which the transmitting medical device has retrieved. The device may either directly be connected with the information system of the hospital, i.e. the electronic patient record, or through suitable image recognition devices, such as medical cameras or scanners or webcams and a following image recognition system for detecting the patient data.

In the case that operating parameters or patient data are only partially transmitted, e.g., due to connection problems, a data set is kept in the device that in such cases is used as a fall-back level and enables a safe operation of the medical device that has sent the data. The central computer computes, on grounds of superordinate rules and/or the evaluation of existing data sets with a clinical result with the incoming data of the medical device from similar data that include an as good clinical result as possible, operating parameters and returns them back to the medical device. One kind of such a calculation may be a simple correlation of the incoming data with the existing data. Thus, the processes being already completed and kept in the memory with the best clinical result under consideration of the superordinate rules are transmitted back as a suggestion for the further operation of the medical device. This procedure is evidence-based, i.e., based on the procedures beforehand successful. Other linkages and calculations according to prior art that provide operating parameters for the further operation of the medical device are also included in the invention.

Further, the central computer groups the incoming data according to certain aspects as, for example, age group, weight, gender and determines the employed medical devices, such as suction pumps and/or in-house suction systems, disposables for fluid transportation, used surgical devices such as shaver or morcellator or RF surgery devices, trocars and optical systems. These are determined either by the properties determined during operation of the medical device by internal sensors, or during the operation of the medical device (intra-operatively) and/or before the operation of the medical device (pre-operatively) via a connection to an electronic surgery planning or surgery accounting system, wherein the used articles are detected for purposes of materials management or accounting and are assigned to the procedure, by an input terminal, e.g., with barcode reader or manual input, by, as may be the case, RFID-coded medical devices, or by a camera-based image recognition.

The communication between the medical engineering apparatus and the medical device may take place through network connections of prior art that may be based either on cable or radiowaves. Usually, such networks transporting sensitive patient data are encrypted and protected against failure. The communication devices may be configured online as a network connection or also offline as a data storage that are physically transported between medical device and medical engineering apparatus and can be read and described by devices at the medical device and the medical engineering apparatus.

The communication may comprise a set value recommendation for the pressure in the body cavity based on patient's age (e.g., gentle mode for children), patient's gender (e.g., abdomen pressure different for men and women), procedure (e.g., pressure requirements for certain joints or different for purely diagnostic and therapeutic procedures) or operating parameters of the medical devices (e.g., pre-pressures at pressure sensor for certain hose-sets or optics-trocar combinations). Furthermore, complete parameter sets or characteristic curve data of identified medical products can be transmitted. This enables, for example, the optimum operation with newly launched medical devices and parameters not known before the setting into operation of the medical device of these new medical devices. By communication, the optimum operating parameters determined elsewhere can be transmitted and are thereafter available to the medical device. Also provided according to the invention is the transmission of intra-operatively determined data that are evaluated by the medical engineering apparatus following newest findings, and further operating parameters or set values for the current operative phase in the meaning of an optimum, i.e. an as good as possible clinical outcome transmitted back to the medical device. These are indicated as a recommendation of the medical device and can easily be adopted.

Further, according to the invention, a communication is enabled between the surgeon judging on the clinical outcome and/or the patient with respect to the post-operatively performed assessments of the clinical outcome (e.g., medical scores). This may, in one embodiment, be made via an input mask, the association with the data previously stored and belonging to the data set being made by means of a specific access code. In another embodiment, the medical device can request the post-operative data assigned in the HIS and forward them to the medical engineering apparatus. Other solutions according to prior art for association with the data are also possible.

The medical engineering apparatus contains a processing device that examines the data to be sent back superordinately to the already described calculation rules and accepts the data according to general conditions (from guidelines or medical expert knowledge) or changes them to other values. These superordinate rules are entered via an input device and can be tested and analyzed by an own functional environment (sandbox according to prior art) that is separated from normal operation.

In one embodiment, the medical engineering apparatus with computer etc. can also be integrated in a medical device. Then, the expert system is part of the fluid pump. In another preferred embodiment, the medical engineering apparatus is a separate device that is connected via a network to the medical devices in the operating room.

A concrete scenario of use of the medical engineering apparatus, called expert system in the following, for the use of a fluid pump for expanding body cavities may be as follows: The pump determines all surgery-related data in the hospital network: reference data of the patient (without name, but with case number) plus anamnesis data being relevant for controlling the devices. The pump sends the classified data to the expert system and obtains operating parameters. These are displayed by the pump as recommendations that can be accepted by the surgeon. The data available in the operating room (technical data of the pump and other devices PLUS vital data, i.e. blood pressure, heart rate, pulse oxymetry data, etc.) are sent with an assignment code (identifier) to the expert system and are recorded there. If applicable, state data (see previous list) are sent in the course of the surgery to the expert system, and operating parameters (recommendations that can be accepted by the surgeon), or hints and warnings from the expert system (which then acts as an assistance system) are sent to the pump. The data directly sent back from the expert system to the pump are evaluated or selected using older pseudonymized and classified/stratified patient data sets on grounds of the best clinical outcome. Medical rules per se also can affect the selection. Later collected, i.e. post-operative data can be supplied to the expert system in two ways—both having their own problems:

1) Transfer of a token or access code to the patient asking him or the surgeon in charge to enter the scores. This input would directly be made in the expert system. A problem is the very low response rate and a risk that only certain groups of patients will confirm (bias, in order avoid this, even more data would have to be collected, before a valid selection could be made), therefore this solution is not favored.

2) Follow-up diagnosis in the hospital by follow-up appointment. The follow-up diagnosis information from the hospital is, for example, kept as a list of “open items” in the pump and examined in the HIS and when available transmitted to the expert system. After expiration of a certain time (e.g., the values for assessment of the clinical outcome should be collected 4 weeks after surgery PLUS input period of another 4 weeks), the request is canceled, if the pump was turned off in that time. The request may, e.g., be active in the hose filling phase, when the pump is busy with the surgery setup and not with patient-critical activities or in surgery pauses (recognition “hose removed”); after “hose removed”, however, there may suddenly be no power, since the device is dismantled.

Furthermore, the uninterrupted connection to the expert system is not secured and further can be interrupted during the surgery. For this purpose, a fall-back level in the pump control is provided, and for the expert system a solution of the (safe) renewed assignment when re-establishing the connection is obtained.

Since the expert system, e.g., is to be provided centrally in a computing center and not in the hospital network environment, in order that as many as possible different data sources are available for a data set selection (no bias, large database), for such a connection of pump to expert system, the hospital network would have to be opened to the internet. This opening may also be made as a VPN connection.

1. A medical engineering apparatus for storing medical data, including
at least one computation unit, consisting of a selection and correlation module, a feed module with self-learning sub-module,
at least one storage unit that stores measurement data and/or patient data obtained via the interface and optionally permanently contains at least one data set as a fall-back level,
at least one interface to at least one other medical engineering apparatus, the interface transmitting measurement data and/or patient data to the storage unit,
at least one interface for reading patient information to at least one information system of the hospital (patient record) and/or an image recognition system for reading patient information,
at least one processing device, in which a priori information present in the storage unit is processed with the information obtained via the interface such that operating parameters calculated according to a predetermined criterion are provided via an interface of another medical engineering apparatus.

2. Medical engineering apparatus according to claim 1, wherein the computation unit contains:
at least one system consisting of hard- and software for data communication for networking the devices in the operating room,
at least one system consisting of hard- and software for data classification or grouping of patient data from the pre-operative phase,
at least one system consisting of hard- and software for determination of the one or more medical devices used for the medical procedure,
at least one system consisting of hard- and software for data transmission to the medical devices used for the medical procedure,
at least one system consisting of hard- and software for determination and transmission of set value recommendations based on a priori knowledge to the one or more medical devices,
and optionally:
at least one system consisting of hard- and software for identification of employed medical devices in the pre-operative phase,
at least one system consisting of hard- and software for determination and transmission of a set of characteristic curves for controller pre-setting based on known a priori knowledge to one or more medical devices used for the medical procedure,
at least one system consisting of hard- and software for data transmission from the intra-operative phase to the database system or the medical engineering apparatus of claim 1,
at least one system consisting of hard- and software for determination and transmission of a set of characteristic curves for controller pre-setting based on intra-operative data to one or more medical devices used for the medical procedure,
at least one system consisting of hard- and software for determination and transmission of set value recommendations based on intra-operative data to one or more medical devices used for the medical procedure,
at least one system consisting of hard- and software for data transmission from the post-operative phase to the database system or the medical engineering apparatus of claim 1,
at least one system consisting of hard- and software for entering rules that originate from the medical expert knowledge or medical guidelines.

3. The medical engineering apparatus according to claim 1, wherein the stored data are a mixture of anonymized or pseudonymized patient data, anamnesis data, procedure and device data or device parameters, post-operative treatment assessments, and procedural rules.

4. The medical engineering apparatus according to claim 3, wherein the fluid pump is an insufflator or a liquid pump.

5. The medical engineering apparatus according to claim 1, wherein at least one interface transmits measurement data of a blood pressure measurement device and/or patient data of an electronic patient record.

6. The medical engineering apparatus according to claim 1, wherein as operating parameters, fluid pressure, fluid flow, and/or fluid temperature are controlled.
